(19) United States
(12) Patent Application Publication
(10) Pub. No.: US 2014/0316315 A1
(43) Pub. Date: Oct. 23, 2014

(54) PROTECTION MEMBER AND CONTACT TOOL

(71) Applicant: Naoto Ohira, Tokyo (JP)

(72) Inventor: Naoto Ohira, Tokyo (JP)

(21) Appl. No.: 14/257,661

(22) Filed: Apr. 21, 2014

(30) Foreign Application Priority Data
Apr. 21, 2013 (JP) 2013-88960
Apr. 4, 2014 (JP) 2014-78258

(51) Int. Cl. A61F 5/01

(52) U.S. Cl. CPC A61F 5/0102
USPC 602/6

(57) ABSTRACT

A protection member which protects at least a portion of the body of a person or an animal, includes: an impact relaxing portion which is changed according to the shape of a part of the person or the animal to be mounted and absorbs an impact; a deformation suppressing member which suppresses deformation of the shape of the protection member and disperses the impact absorbed by the impact relaxing portion; and a close contact surface which is provided in at least a portion of a mounting surface of the protection member. The deformation suppressing member includes base bodies made of a material harder than the impact relaxing portion and shape defining portions made of a material harder than the base body. The base bodies are provided to be lined up along the planar direction of the protection member, and the shape defining portion is provided in at least a portion between the base bodies.

### BACKGROUND OF THE INVENTION

1. Field of the Invention

The present invention relates to a protection member which protects the body of a person or an animal, and a contact tool.

2. Description of the Related Art

Hitherto, many supporters for protecting the body of a person have been suggested. As the supporter, a supporter in which an elastic member is applied on the outside and a member having cushioning properties is applied on the inside is suggested (refer to JP 2003-164484 A).

### CITATION LIST

### Patent Literature

Patent Literature 1: JP 2003-164484 A

The invention provides a protection member capable of effectively protecting the body of a person or an animal and a contact tool.

### SUMMARY OF THE INVENTION

### 1. Protection Member

A protection member of the present invention for protecting at least a portion of the body of a person or an animal, includes: an impact relaxing portion which is changed in shape according to a shape of a part of the person or the animal to be mounted, and absorbs an impact; a deformation suppressing member which suppresses deformation of a shape of the protection member and disperses the impact absorbed by the impact relaxing portion; and a close contact surface which is provided in at least a portion of a mounting surface of the protection member that is mounted on the person or the animal. The deformation suppressing member includes a base body made of a material harder than the impact relaxing portion and a shape defining portion made of a material harder than the base body.

According to the protection member according to one or more embodiments of the present invention, an impact can be absorbed by the impact relaxing portion, the deformation of the protection member itself can be suppressed by the deformation suppressing member, and the impact can be dispersed. Accordingly, a change in the shape of the protection member is suppressed, and a burden on a part to be protected can be reduced. In addition, a change in the shape of the part to be protected can be suppressed, and the shape of the part to be protected can be secured. Since the deformation suppressing member includes, in addition to the base body, the shape defining portion made of a material harder than the base body, a function of maintaining the shape of the deformation suppressing member can be enhanced.

In one or more embodiments of the present invention, a plurality of the base bodies may be provided, the plurality of base bodies may be provided to be lined up along a planar direction of the protection member, and the shape defining portion may be provided in at least a portion between the base bodies. Accordingly, the shape defining portion can be provided to be interposed between the base bodies, and thus a function of maintaining the base bodies can further be enhanced. That is, since the base bodies are provided to be lined up along the planar direction of the protection member and the shape defining portion is provided in at least a portion between the base bodies, the deformation of the deformation suppressing member can be accurately suppressed. Further, an applied pressure can be accurately dispersed by the shape defining portion, and the part to be protected can be effectively protected.

In one or more embodiments of the present invention, the shape defining portions and the base bodies may be alternately arranged. Accordingly, an applied pressure can be more accurately dispersed by the shape defining portions.

In one or more embodiments of the present invention, a cross-sectional shape of the deformation suppressing member may be a cantilever shape or a bridge shape. In a case where the deformation suppressing member has the cantilever shape or the bridge shape, thinner parts than other parts can be generated. However, compared to a case of only the impact relaxing portion, an impact applied to the part to be protected can be suppressed.

In one or more embodiments of the present invention, the shape defining portions may be configured of element members of the plurality of shape defining portions, and the element members of the plurality of shape defining portions may be arranged in a row. Accordingly, in the shape defining portions, a degree of impact dispersion can be increased.

In one or more embodiments of the present invention, an extension direction of the element members of the shape defining portions may be an oblique direction with respect to a thickness direction of the protection member. Accordingly, in the shape defining portions, a degree of impact dispersion can be increased.

In one or more embodiments of the present invention, a groove may be provided on a surface of the shape defining portion. Accordingly, in the shape defining portion, a degree of impact dispersion can be increased.

In one or more embodiments of the present invention, an extension direction of the groove may be an oblique direction with respect to a thickness direction of the protection member. Accordingly, in the shape defining portions, a degree of impact dispersion can be increased.

In one or more embodiments of the present invention, the deformation suppressing member may be made of a material having a heat retention or a cooling function. Accordingly, the part protected by the protection member can be kept warm or be cooled.

In one or more embodiments of the present invention, an accommodation portion which accommodates a cooling material or a heat retention material may further be included. Accordingly, the cooling material or the heat retention material can be easily accommodated.

A contact tool of one or more embodiments of the present invention is provided with the protection member on the surface.

### BRIEF DESCRIPTION OF THE DRAWINGS

FIG. 1A is a plan view of a protection member according to an embodiment, and FIG. 1B is a diagram schematically illustrating the cross-section of the protection member taken along line A-A of FIG. 1A.

FIG. 2 is a diagram schematically illustrating a shape defining portion.

FIG. 3A is a diagram schematically illustrating element members of the shape defining portion, and FIG. 3B is a diagram schematically illustrating the shape defining portion configured by assembling the element members of the shape defining portion in a row.

FIG. 4 is a diagram schematically illustrating a layer structure of a deformation suppressing member.

FIG. 5A illustrates a form of the protection member when being mounted as a knee supporter, and FIG. 5B is a diagram schematically illustrating a surface of a side where a close contact surface is provided when the protection member is deployed.

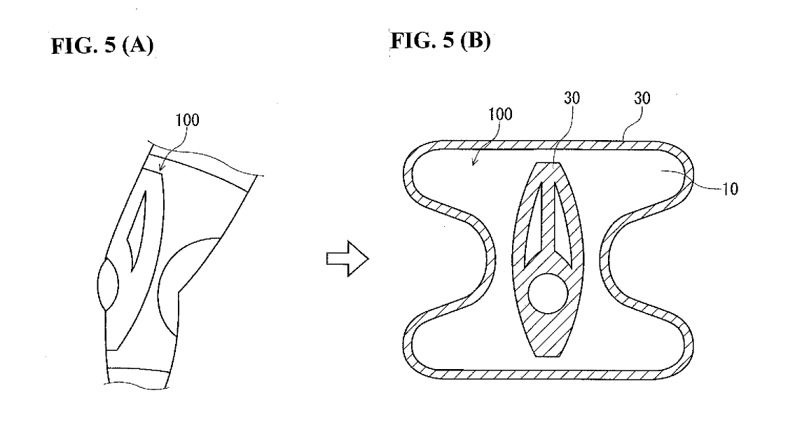

FIGS. 6A and 6B are diagrams illustrating an action due to a difference in the shape of the protection member.

FIG. 7 is a diagram illustrating an action of the protection member.

FIG. 8 is a diagram schematically illustrating a modified example of the protection member.

FIG. 9 is a schematic diagram of a contact tool in a case where a chair is exemplified as the contact tool.

FIG. 10 is a diagram schematically illustrating a modified example of the protection member.

FIG. 11 is a diagram schematically illustrating a modified example of the protection member.

FIG. 12 is a diagram schematically illustrating a modified example of the protection member.

### DETAILED DESCRIPTION OF THE PREFERRED EMBODIMENTS

Hereinafter, exemplary embodiments of the invention will be described with reference to the drawings.

### 1. Protection Member

A protection member 100 protects at least a part of the body of a person or an animal. The protection member 100 includes an impact relaxing portion 10 which absorbs an impact, a deformation suppressing member 20 which suppresses the deformation of the impact relaxing portion 10, and a close contact surface 30 which is provided in at least a part of a mounting surface of the protection member 100 mounted to a person or an animal.

The impact relaxing portion 10 absorbs an impact added to the protection member 100 and is changed in shape along the shape of a part to be mounted. The impact relaxing portion 10 may be interposed between the deformation suppressing member 20 and the close contact surface 30. Accordingly, a pressure of the deformation suppressing member 20 on the part to be mounted can be relaxed and an uncomfortable feeling during mounting can be reduced. Since the impact relaxing portion 10 is changed in shape along the shape of the part to be mounted, a gap from the body is filled and thus stability is provided.

The impact relaxing portion 10 is not particularly limited as long as it has an impact absorbing function, and a well-known bubble cushioning material, a liquid bubble cushioning material made of a liquid such as water, a well-known gel-like material having impact absorbing performance may be applied. The impact relaxing portion 10 may be made of a material having a heat accumulating action, for example, polyethyleneglycol or a water-absorbing polymer (polyacrylic acid-based) material. By disposing the impact relaxing portion 10 to directly come into contact with the skin, the material is warmed by body heat and a heat retention effect can also be maintained.

The impact relaxing portion 10 may be provided on the side of a part of a body to be protected or may also be provided on the outside of the opposite side to the side of the body to be protected.

By providing the impact relaxing portion 10 on the side of the part to the protected, the impact absorbing material may be installed close to the body. By absorbing an impact (weight or the like) from the inside of the body, the impact that returns to the body or an impact on the body against the ground or the like can be reduced.

In addition, since the impact relaxing portion 10 is provided on the outside (the opposite side to the side of the part to be protected), an external impact (an impact from a collision with a tackle, a ball, or the like) can be absorbed. That is, by disposing the impact relaxing portion 10 (an air layer or the like) on the outside, the impact is dispersed and thus the transmission of the impact to the inside can be minimized.

A heat retention member or a cooling member may be provided inside the impact relaxing portion 10. The impact relaxing portion 10 may be made of a heat shielding material or a heat insulating material, for example, a bubble cushioning material to be able to enhance a heat shielding or heat insulating function. In addition, by forming the shape of polystyrene foam into beads shape, the polystyrene foam may be inserted into the space of the bubble cushioning material. The thickness of the impact relaxing portion 10 is not particularly limited, and for example, may be set to 1 mm to 1 cm. The impact relaxing portion 10 may not have the same hardness, and may have partially different hardness.

The heat retention member or the cooling member may be provided in the protection member 100 to be divided. In this case, only the heat retention member or the cooling member can be heated or cooled, and thus heating or cooling the entire impact relaxing portion 10 can be avoided. In addition, since the heat retention member or the cooling member is provided in the protection member 100 to be separated therefrom, a necessary layer can be added later. Since the cooling member or the heat retention member can be added, the usage range or the purpose of use can be significantly widened, for example, icing (treatment) or warming can be performed while supporting muscle. Instead of the heat retention member or the cooling member, a protection layer capable of protecting a body part damaged by contusion or the like may be added to a supporter for supporting muscle. A close contact surface may also be provided to each of contact surfaces of the impact relaxing portion 10 that come into contact with the heat retention member and the cooling member. The close contact surface may have the same configuration as that of a close contact surface 30, which will be described later.

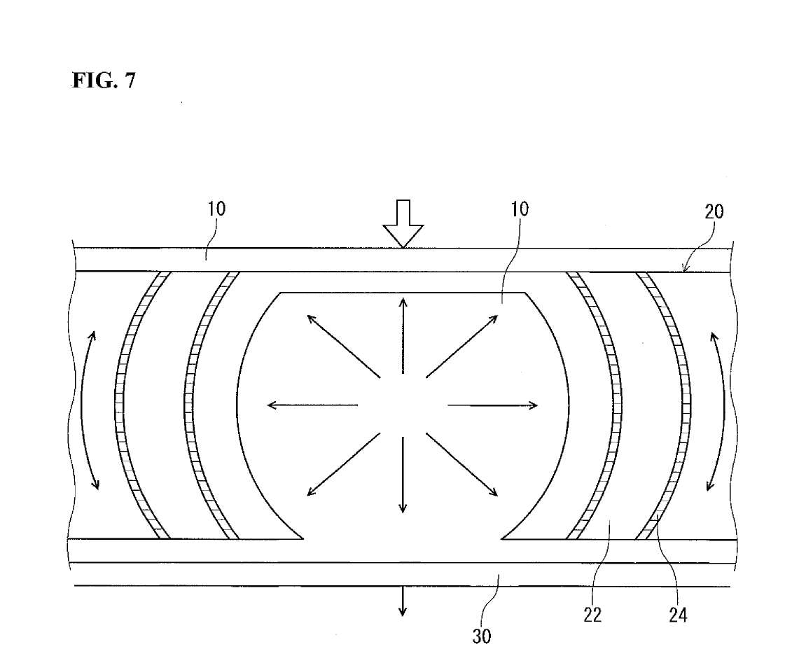

As illustrated in FIG. 7, the deformation suppressing member 20 has a role of suppressing the deformation of the shape of the protection member 100 and dispersing the impact absorbed by the impact relaxing portion 10. The deformation suppressing member 20 may be provided at a position adjacent to the impact relaxing portion 10. The deformation suppressing member 20 may have a function of holding the shape of the protection member 100. By the deformation suppressing member 20, the structure of a human body or the like can be efficiently supported. In addition, the deformation suppressing member 20 may be provided to be enclosed by the impact relaxing portion 10, may be provided to overlap the impact relaxing portion 10 in the thickness direction or the planar direction, or may be provided in the vicinity of the impact relaxing portion 10. By providing the impact relaxing portion 10 on the side of the body in relation to the deformation suppressing member 20, an uncomfortable feeling during mounting or movement is reduced.

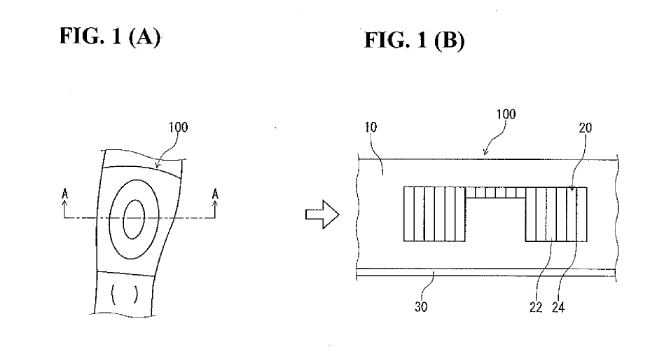

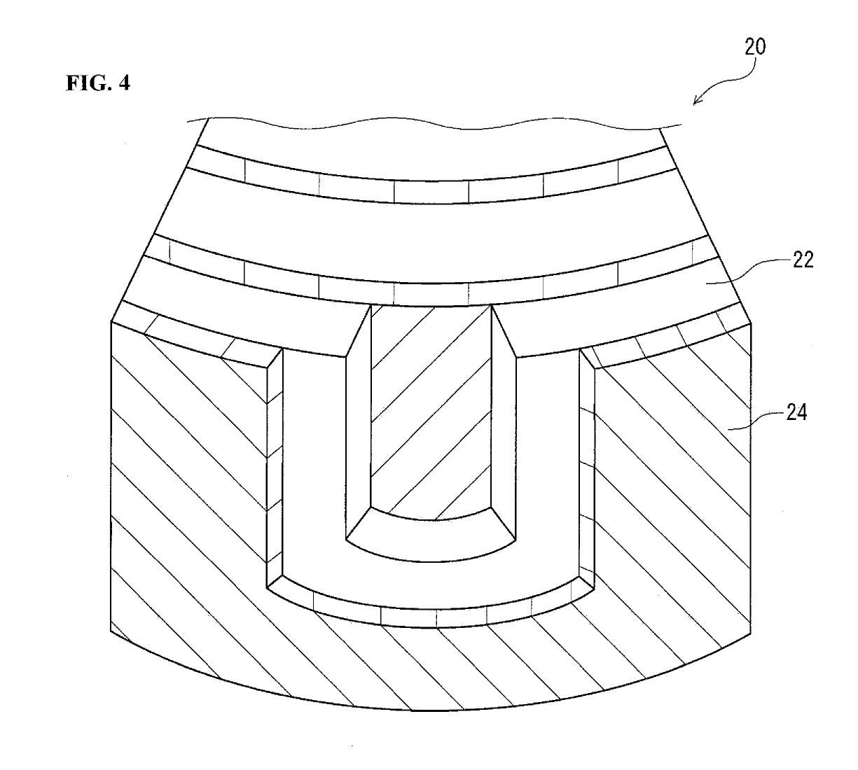

As illustrated in FIGS. 1 and 4, the deformation suppressing member 20 includes a base body 22 and a shape defining portion 24. The base body 22 and the shape defining portion 24 may be provided to be lined up along the planar direction of the protection member 100. The base body 22 may be made of a material harder than the impact relaxing portion 10. A single or a plurality of the base bodies 22 may be provided. The shape defining portion 24 may be made of a material harder than the base body 22. A single or the plurality of shape defining portions 24 may be provided. In the case where the plurality of base bodies 22 are provided, as illustrated in FIGS. 1A and 1B, the base bodies 22 may be provided to be lined up along the planar direction of the protection member 100 at predetermined intervals. The shape defining portion 24 may be provided in at least a part between the base bodies 22, and for example, may be provided in a form interposed between the base bodies 22. That is, the shape defining portions 24 and the base bodies 22 may be alternately arranged. Accordingly, an added pressure can be more accurately dispersed by the shape defining portions 24.

The deformation suppressing member 20 may be made of, for example, a fibrous member. Since the deformation suppressing member 20 is made of a fibrous member, the thickness of the deformation suppressing member 20 can be reduced. By arranging the base bodies 22 to be separated from each other, the pressure can be dispersed and at the same time, a necessary degree of elongation of the base bodies 22 can be ensured.

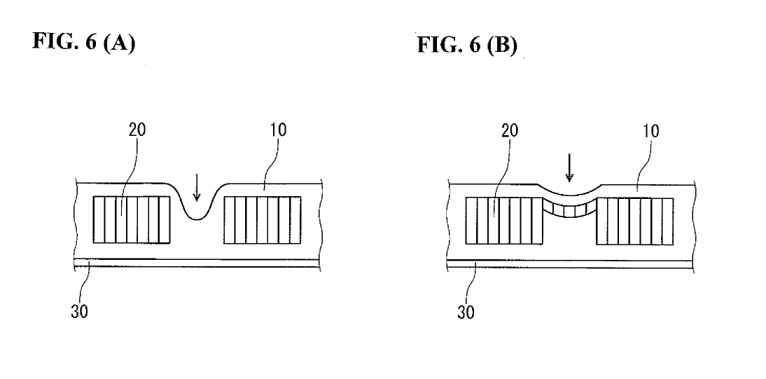

The shape of the deformation suppressing member 20 is not particularly limited, and the cross-sectional shape thereof may be a shape having thin parts and thick parts such as a cantilever shape or a bridge shape. Accordingly, as illustrated in FIGS. 6A and 6B, the impact can be dispersed by the thin parts (for example, a bridge part) of the deformation suppressing member 20 while ensuring the impact absorbing function of the impact relaxing portion 10, and thus the impact is less likely to reach the part to be protected.

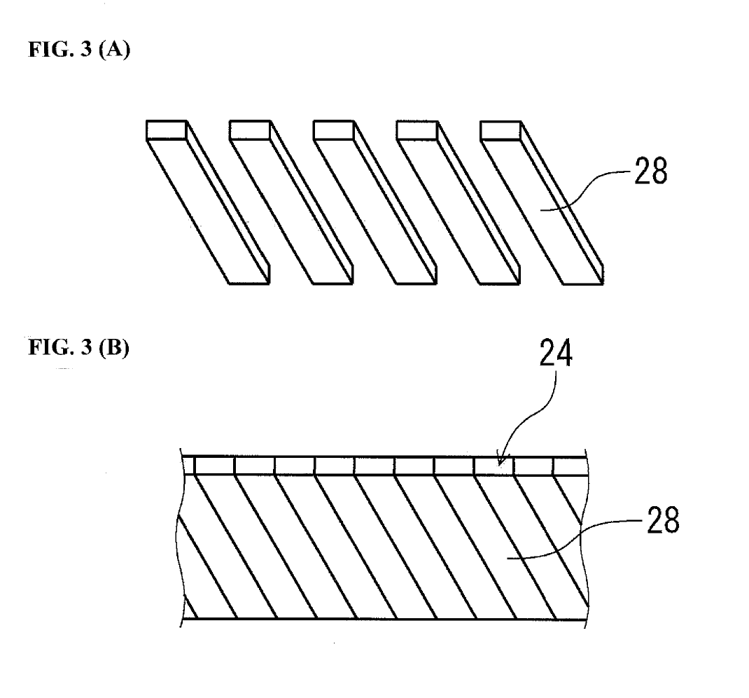

As illustrated in FIG. 3A, the shape defining portion 24 may include a plurality of element members 28. That is, the shape defining portion 24 may be configured from an assembly of the plurality of element members 28. The shape of the element member 28 may be a rectangular parallelepiped such as a quadrangular prism or a columnar body such as a parallelogram, and the opposing surfaces thereof such as the upper surface and the lower surface may not be parallel to each other. The element members 28 of the shape defining portion 24 may be provided in a row as illustrated in FIG. 3B. The elongation direction of the element member 28 of the shape defining portion 24 may be an oblique direction with respect to the thickness direction of the protection member 100. The extension direction of the element member 28 of the shape defining portion 24 may be set to a direction intersecting the extension direction of the element member 28 of the shape defining portion 24 in the adjacent shape defining portion 24. Accordingly, the shape maintaining function of the shape defining portion 24 can be enhanced, and the impact dispersing function of the shape defining portion 24 can be enhanced.

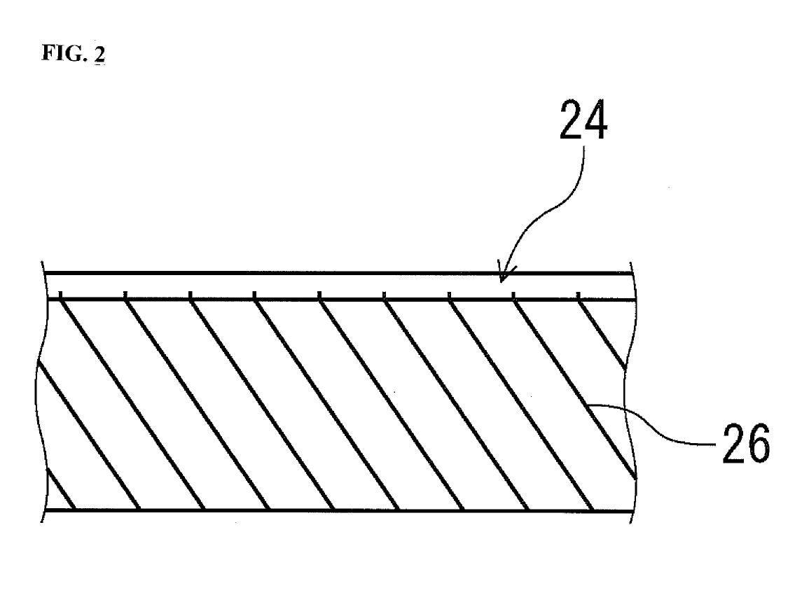

As illustrated in FIG. 2, the shape defining portion 24 may be provided with a groove 26 on the surface of the shape defining portion 24. The extension direction of the groove 26 may be an oblique direction with respect to a predetermined direction, for example, the thickness direction of the protection member 100. The extension direction of the groove 26 may be set to a direction intersecting the adjacent shape defining portion 24. As such, by forming the grooves 26 provided in the respective shape defining portions 24 adjacent to each other to have different extension directions, not only a pressure in one direction can be dispersed, but also pressures in multiple directions can be coped with. In addition, shape maintenance can be efficiently achieved while maintaining elasticity of the deformation suppressing member 20. In addition, in the example illustrated in FIG. 2, the case where the grooves 26 are formed in a row is illustrated. However, the grooves 26 are not necessarily provided in parallel as long as they can disperse an impact, and for example, may be formed in a wave pattern.

By providing the grooves 26 in the oblique direction on the surface of the shape defining portion 24, the shape maintaining function of the shape defining portion 24 can be enhanced, and an ability to disperse an impact can further be enhanced. That is, by providing the grooves 26, a pressure can be dispersed in a direction in which the grooves 26 are provided. In addition, since the shape defining portion 24 uses a material harder than the base body 22, an extra extension can be suppressed. Therefore, the stabilization of the structure can be achieved while minimizing the collapse of the shape. When a pressure is applied to the protection member 100, a tension occurs in the shape defining portion 24. However, since the grooves 26 are provided, the direction in which the tension is applied is determined, and the tension stabilizes the structure, thereby supporting shape maintenance and the body structure. The shape of the shape defining portion 24 may be formed in a ring shape such as an annual ring or annulus fibrosus shape in the deformation suppressing member 20. Since the shape defining portion 24 is provided, a force to hold the shape of the deformation suppressing member 20 itself can be increased, and a pressure applied to the deformation suppressing member 20 can be dispersed. Since the deformation suppressing member 20 is provided, the structure is stabilized, and the pressure is dispersed, thereby efficiently supporting the body structure.

The deformation suppressing member 20 may be configured of a material having a heat retention function or a heat absorbing function. Accordingly, a part of a person or an animal covered with the protection member 100 can be kept warm or be cooled.

The base body 22 and the shape defining portion 24 of the deformation suppressing member 20 may be made of a material having elasticity such as silicon, urethane, or neoprene, an elastic fiber such as polyurethane, or a well-known material having a shape-memory function. The shape defining portion 24 may be made of a material harder than the base body 22, or may be made of the same type of material as the base body 22 with a different hardness or a different type of material from the base body 22 with a different hardness.

The deformation suppressing member 20 is not particularly limited as long as it is made of a material harder than the impact relaxing portion 10, and for example, on the premise that the deformation suppressing member 20 may be made harder than the impact relaxing portion 10, the material of the impact relaxing portion 10 described above may be applied.

The shape of the deformation suppressing member 20 may be a shape along the unevenness of the part to be mounted. Accordingly, since the shape of the deformation suppressing member 20 is adapted to the shape of the body site, the site can be supported accurately, and furthermore, a shift due to movement or an uncomfortable feeling during mounting can be reduced.

By increasing or decreasing the number of shape defining portions 24, the thickness thereof, or the width of the interval therebetween, the elongation rate that is generated when a weight is applied or during a stretch caused by movement or the strength of the deformation suppressing member 20 can be controlled. When the interval between the shape defining portions 24 is narrowed, the elongation rate can be relatively reduced, and the interval therebetween is increased, the elongation rate can be relatively increased. In addition, a function of holding elongation by the base body 22 and preventing excessive elongation by the shape defining portion 24 may be imparted.

The shape defining portion 24 may have the same hardness in each part or may have partially different hardnesses. In the same manner, the base body 22 may also have the same hardness in each part or may have partially different hardnesses.

The impact relaxing portion 10 and the deformation suppressing member 20 may be provided to be divided or separated from each other. Specifically, a space is provided inside the impact relaxing portion 10, and the deformation suppressing member 20 may be accommodated in the space to be taken out. A configuration which allows the impact relaxing portion 10 and the deformation suppressing member 20 to come into close contact with each other may be employed between the impact relaxing portion 10 and the deformation suppressing member 20. The configuration which allows a close contact may apply the same material as that of the close contact surface 30, which will be described later. By providing the impact relaxing portion 10 and the deformation suppressing member 20 to be dividable or separable, adjustment of intensity can become easy. In addition, this embodiment can become easy to establish the deformation suppressing member 20 to the different plural layers depending on the purpose, and the deformation suppressing member 20 can be customized to suit a wide variety of purposes. The deformation suppressing member 20 can also be adjusted the intensity depending on the purpose. This embodiment can be exchanged only the layer that deteriorated. By exchanging the core layer into the object in accordance with the shape of other body parts, this embodiment can be adopted at multiple body parts in one product. By adjusting the color of each layer or member, this embodiment can be provided the various designs, and can be exchange the layer for design. By changing the combination of the impact relaxing portion 10 and the deformation suppressing member 20, this embodiment can be adopted to a wide variety of protectors. The protective layer can be added to the required location as necessary.

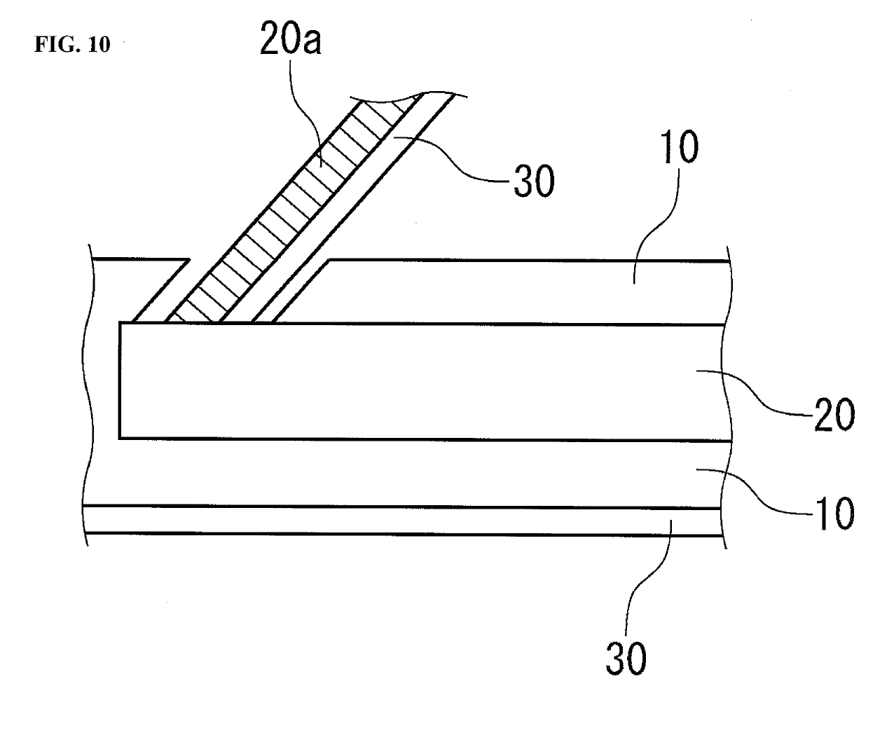

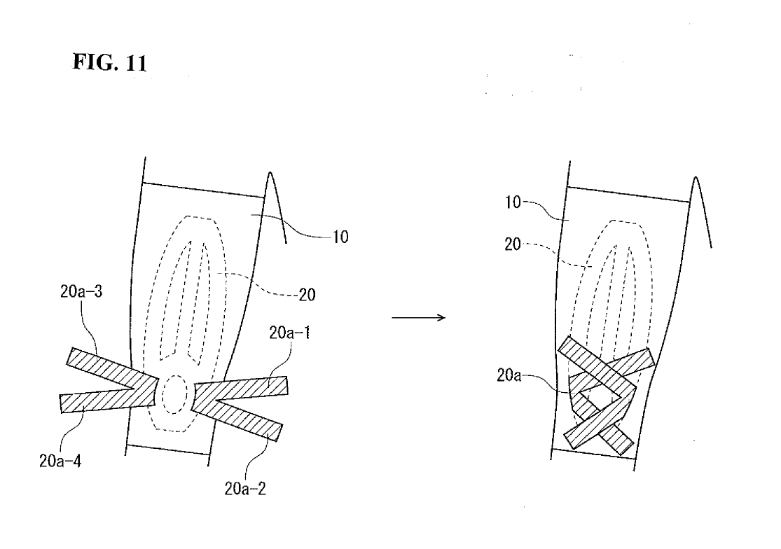

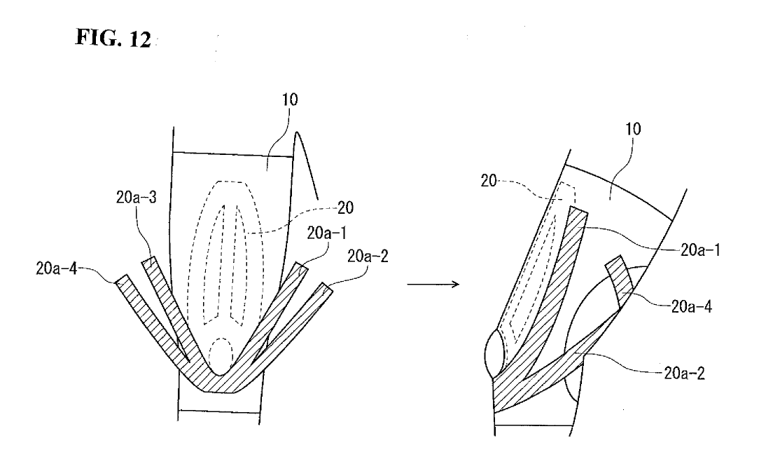

As illustrated in FIGS. 10 and 11, the deformation suppressing member 20 may have the projecting portion 20a that protrudes the impact relaxing portion 10 towards outside. Furthermore, the projecting portion 20a may have the close contact surface 30 that can be mounted to a person or an animal. As illustrated in FIG. 10, the deformation suppressing member 20 may have the ramification structure. The number of ramification may be a single or multiple. The structure to be branched may also be further branched. By extended the projecting portion 20a to the outside so as to penetrate the impact relaxing portion 10, this embodiment can be provided further support after mounting, and can become easy to adjust the intensity of support. The projecting portion 20a can be branched from the base of the deformation suppressing member 20 or from the other parts of the deformation suppressing member 20 for supporting the structure. The projecting portion 20a can be mounted to overlap with the protection member 100 for enhancing the ability of support. In addition, the projecting portion 20a can be provided a more extensive support by changing the support direction when mounting. The projecting portion 20a can be provided the one directional support, but also the multidirectional support. By making the projecting portion 20a plurality, a combination of various supports can be accomplished. As illustrated in FIG. 11, the projecting portion 20a-1 and 20a-2 extending from the one side and the projecting portion 20a-3 and 20a-4 extending from the other side may be mounted so as to intersect on the protection member 100. As illustrated in FIG. 12, the projecting portion 20a may be mounted so as to go around the back of the body part where the protection member is mounted.

A heat retention function or a cooling function may be imparted to the impact relaxing portion 10 or the deformation suppressing member 20. For example, a heat retention member employs a structure including bubbles to be warmed by a microwave and thus can be easily heated to a predetermined temperature. As the material of the heat retention member, a material that gradually transfers heat to the body, a material that emits far-infrared rays, and the like may be applied. As the material that gradually transfers heat to the body, for example, a silicone rubber or water (for example, gel-like water) may be applied. As the material that emits far-infrared rays, for example, a material (for example, Sansen stone or black serpentine) that emits far-infrared rays in a waveband (photoelectron grow light) of 4 to 14 microns is preferable because the material has little stimulus to the skin and is most absorbed by the human body.

A deodorizing, antibacterial, dampproofing, or moldproofing function and the like may be imparted to the impact relaxing portion 10 or the deformation suppressing member 20.

In general, in a case where the protection member 100 is attached after applying an antiphlogistic cream, the degradation in adhesion is considered. Therefore, a medicated component such as an antiphlogistic agent may be kneaded into the impact relaxing portion 10 or the deformation suppressing member 20.

The close contact surface 30 may be determined in consideration of a required adhesion holding force or an ease of detachment and may select a material from well-known adhesive members. The close contact surface 30 may be provided on the entirety of a surface where the protection member 100 directly or indirectly comes into contact with the body of a person or an animal or may be provided on a part of the surface.

The close contact surface 30 has a close contact structure on a surface for a human body structure or those (for example, clothes) that come into contact with the human body structure. The close contact surface 30 is not particularly limited as long as it has a close contact function and may be configured of an adhesive, or may also be configured of an adsorption surface or a surface made of a material having a high coefficient of friction. A support site as an object can be accurately supported by the close contact surface 30 without a shift therefrom. In addition, by allowing the close contact surface 30 to come into close contact, an unnecessary mounting part can be reduced and a pressurizing sensation can be reduced. In addition, since the close contact surface 30 can be mounted only to the site as an object, impediment to movement is minimized.

The surface of the close contact surface 30 may be an uneven surface (an irregular surface having a wrinkle shape, wavelike shape, projection shape, or the like). In this case, the uneven surface of the close contact surface 30 makes the surface of the impact relaxing portion 10 uneven, and an adhesive may be provided along the surface thereof. In addition, the surface of a base member such as a sheet of the close contact surface 30 may be made uneven and may be configured so that an adhesive is provided along the surface thereof. In a case where the uneven surface is provided as such, compared to a case where an adhesive is formed on a flat surface, the uneven surface is effective in adjusting the adhesion.

In the case where the adhesive is used, the arrangement pattern of the adhesive is set to a wave shape instead of arranging the adhesive over the entire surface, and thus an area that directly comes into contact with the human body is reduced, thereby further avoiding a problem such as an allergic reaction. In addition, in consideration of air permeability and an ease of detachment, the uneven surface is advantageous.

The protection member 100 may have a shape along the structure of muscle of the part to which the protection member 100 is mounted. Accordingly, adjustment of a tension in a muscle contraction direction, fine adjustment in a contraction direction, adjustment of a skeletal alignment by muscle function improvement, adjustment of a muscle function, and the like can be performed, and a taping function may be imparted.

Although the constituent material of the protection member 100 is described above, a material may be considered from various viewpoints of air permeability, water repellency, stretchability, strength, service life, body tension properties, and the like. An antimicrobial or deodorizing function may be imparted to the protection member 100.

The thickness of the protection member 100 is not particularly limited and is arbitrarily set depending on a required function, and for example, may be set to 1 to 5 cm. The planar shape of the protection member 100 may be a line shape other than a surface shape.

The impact relaxing portion 10 and the deformation suppressing member 20 may be formed by attaching each member. In addition, the impact relaxing portion 10 and the deformation suppressing member 20 may be formed by coating or printing. In addition, the impact relaxing portion 10 and the deformation suppressing member 20 may be formed by performing coating or printing a plurality of times. Adjustment of thickness is easily performed by coating or printing.

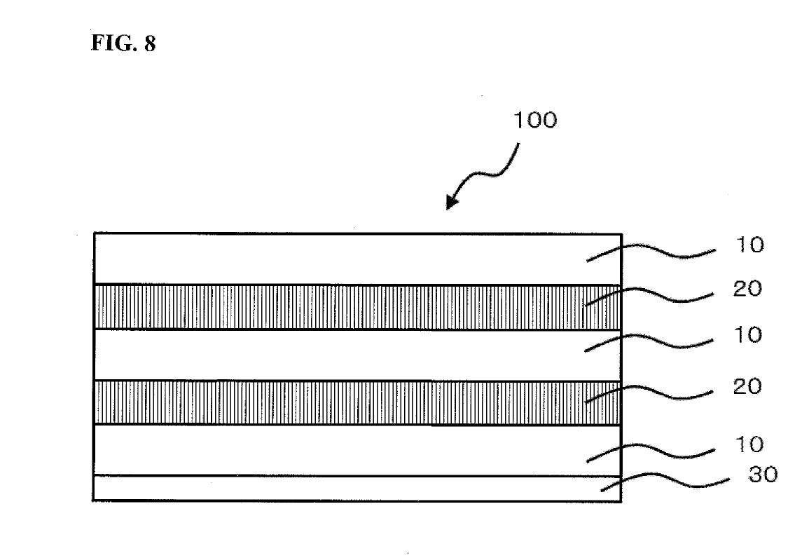

As illustrated in FIG. 8, the protection member 100 may have a structure in which the impact relaxing portion 10 and the deformation suppressing member 20 are laminated. In addition, the impact relaxing portions 10 and the deformation suppressing members 20 may alternately overlap. The structure in which the impact relaxing portions 10 and the deformation suppressing members 20 are laminated and the structure in which the deformation suppressing members 20 are enclosed by the impact relaxing portions 10 may be combined.

The center part of the protection member 100 may be softer than other parts. By configuring the center part to be softer than other parts as such, a degree of impact absorption may be increased by the center part of the affected area. In addition, by forming the close contact surface 30 side to be softer than the outside parts, an uncomfortable feeling can further be reduced when the protection member 100 is attached to the affected area.

### 2. Action Effect

The protection member 100 according to this embodiment can correct the body to its original right position and can continuously support the body.

In addition, according to this embodiment, compared to existing supporters, the deformation suppressing member 20 can define the shape of the protection member 100 and disperse an impact (pressure). In addition, adjustment of a tension, partial addition of a pressure, positional adjustment of a support site, and the like can be performed by the impact relaxing portion 10, and thus slight differences in individuals such as persons or entities can be coped with.

Compared to typical taping, advantages such as a reduction in an uncomfortable feeling during mounting, enhancement in processing performance such as waterproofing, improvement in a difficulty in detachment, and prevention of damages caused by a taping element (fiber such as cotton is used as a product according to the conventional products) rubbed on the skin (particularly the sole or heel parts) can be imparted.

Since mounting parts are mounted by the close contact surface 30, there is no need to additionally provide a member for fixing, handling is easily performed, and there is a small limitation on the range of movement.

The protection member 100 according to this embodiment can define the movement of muscle and promote necessary movements. Cushioning properties for a part that needs to be supported can be ensured by the impact relaxing portion 10. Since the mounting surface of the protection member 100 follows the shape of the body, attachment is easily performed, and handling is easily performed without professional knowledge about taping or supporting.

The protection member 100 may also be attached to the affected area directly without limiting an existing range of joint motion. The protection member 100 may not be necessary to go around a body part because the protection member 100 has the close contact surface 30. Since the protection member 100 can be covered with only the affected area, an unnecessary sensation of compression can be eliminated.

Taping structure can play an original role for taping because a target site can be accurately supported by the existence of the close contact surface 30.

The protection member 100 may also be provided the role such as the poultice by adding the layer including antiphlogistic agent. The combination of taping and the poultice that may not be used at the same time can be accomplished.

By adding the layer including the cooling material or a heat retention material to the protection member 100 that has taping structure, the effects from cryotherapy or thermotherapy can also be expected at the same time.

The protection member 100 can be formed by assembly of lines instead of covering of the surface shape. Assembly of lines can be achieved the same effects as the surface shape of the protection member 100. The protection member 100 may also be provided easy adjustment of intensity by changing the density of assembly of lines. By assimilating into design and decoration parts, supporter parts may be allowed to be invisible. By adding the ramification structure, the protection member 100 can be provided the support in wider range and to multiple directions.

### 3. Contact Tool

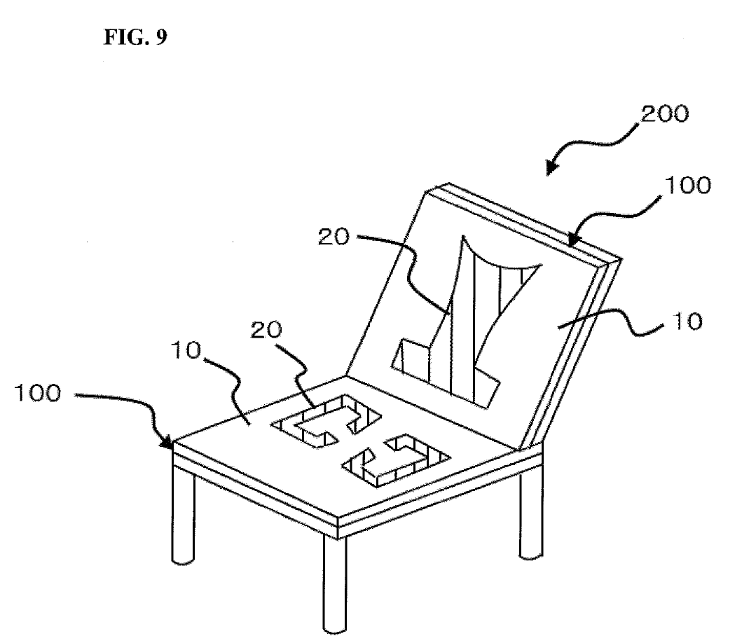

The protection member 100 may also be provided on the surface of a contact tool of a person or an animal. The protection member 100 may be provided to cover the surface of a part of the contact tool or the entire surface thereof. The contact tool is not particularly limited as long as the contact tool comes into contact with the body of a person or an animal. In a case where the contact tool is, for example, a chair, as illustrated in FIG. 9, a contact tool 200 may be configured by providing the protection member 100 on a side which comes into contact with the body of the chair. By applying the protection member 100 to chair and seats, supporting layer that has a sufficient hardness for supporting the body and flexible layer that covers supporting layer can mitigate the discomfort to the body by adjusting the hardness enough to support the body into optimal condition that is not too hard or too soft. The combination of the advantages of both hardness and softness can become important factor in order to improve the comfort and to enhance the fitting to the body. This embodiment can be utilized for variety of products such as a small product like a cushion, a sofa, seat of vehicle, seat of airplane, and seat of rollercoaster in amusement park.

Examples of the contact tool may include grips (handles of vehicles or bicycles, training apparatuses, tools, handrails (for fall prevention in bathrooms, stairs, or the like), umbrellas, sticks, writing tools), seatbelts, straps of backpacks or parts thereof corresponding to the back, nose pads or sides of glasses, goggles, earplugs, earphones, headphones, mouthpieces, wigs, false eyelashes, belts of watches, heart rate monitors (including chest attached type), grips of crutches and parts corresponding to the armpit, manual sections of wheelchairs, child safety seats, straps of flip-flops, toilet seats, tapes for taping (reusable), dog collars, dog shoes, saddles, lids of caps of bottles, bulletproof vests, parts of a rifle that come in contact with a human body (for example, strap parts), insoles, wetsuits, bedclothes, seating surfaces and backrest parts of chairs, seating surfaces (saddles) of vehicles and bikes, floors of bathrooms, gloves (for combat sports such as boxing, for ball games such as baseball, substitutes for bandage for protecting fists, for golf, for football, and the like), dummy targets used in combat sports and contact sports such as punching bags and mitts, ski boots, swimsuits (high-speed swimsuits), nasal plugs (synchronized swimming and the like), belly bands, socks (for those usually used, for swelling prevention, and the like), pads for bedsore prevention, handles of bags and the like, triangular bandages, therapy implements (seating surfaces, back parts, and massage balls of massage chairs, acupressure sticks, and the like), beauty supplies (facial rollers, face masks, and the like), artificial joints, and the like.

### 4. Specific Application Example

### (a) High-Performance Wear

The protection member 100 may be applied to high-performance wear such as training wear and the like. In a case where supporters or taping cannot be used due to the appearance of stage outfits, by integrating costumes into supporters (taping) or assimilating the supporters into design, the supporters can be used without taking care of the physical appearance. A possibility of wearing on the arm and the trunk, separated wearing on the front and the rear of the trunk, and combined wearing on only the chest and only the belly may be considered. By adding a taping function, functions can be easily added depending on the purposes (swelling prevention, pelvis tightening, posture improvement, fall prevention, and the like).

The conventional products may be created the limitation of muscle action by the compression at the time of wearing tightly. Accordingly, the products may be increased the caloric consumption by increasing the loads to the muscles. To add compression to the muscles can give a certain stimulation to the muscles. The conventional products are added a fast dry function, improved air permeability, and thermal insulation function depending on the fibers to be used.

In this embodiment, the protection member 100 has the structures for the purpose of enhancing muscle function without adding excess load to the muscles. By the existence of the close contact surface 30, this embodiment can be mounted to the shape of the body.

This embodiment can be provided the compression to the muscles as in the conventional products. Moreover, by adding the taping structure, the protection member 100 can be promoted optimal muscle contraction without compression, and the protection member 100 can be effectively supported the function that the muscle originally has.

This embodiment can be provided extra functions by fibers to be used, and can be provided a variety of functions by adding a layer conforming to the purpose (the layer including cooling material, heat retention material, weight, antiphlogistic agent and the like). The protection member 100 can be made of fibrous materials, and can also be made of a material having elasticity such as silicon.

The protection member 100 can be changed the muscle (or muscle group) to be supported by the shape of the layer to add, and the purpose of use can thereby be diversified (including the improvement of pelvic adjustment, prevention of swelling, improvement of the muscle balance, and the like).

In a case where supporters or taping cannot be used due to the design of costume or stage outfits, by integrating costumes into supporters (taping), assimilating the supporters into design, or adding the support layer inside of the costume, the supporters can be provided the support to the body structure and the enhancement of the body function without taking care of the physical appearance. A possibility of wearing on only the arm, only the trunk, separated wearing on the front and the rear of the trunk, and the selective wearing to the necessary part may be possible.

### (b) Support by Surface and Support by Assembly of Lines

There may be a case where the protection member 100 is formed in a shape having a width such as a tape and muscles are supported by covering the muscles with the surface thereof, and muscles can also be supported by arranging several line-shaped structures to be lined up.

By an assembly of lines, using the assembly as a part of design while achieving support may also be considered. By adding the same line pattern, supporter parts may be allowed to be invisible. The protection member 100 may also be provided easy adjustment of intensity by changing the density of the linear support material.

### (c) Correction Underwear

The protection member 100 may be applied to correction underwear. Not only the physical appearance is simply changed through tightening, the return to the original body shape after detachment is avoided. Therefore, an efficient muscle action is retrained through wearing and the corrected body shape is maintained even after the detachment, thereby achieving supporting.

The correction underwear is for the fixation of the physical appearance, and the conventional products have the function for tightening and pulling the muscles or body fat. Those effects are a function of the product itself, and the physical appearance may return to the original body shape after detachment because there is no difference of the body function between before and after wearing of the correction underwear.

However, for correcting the shape of the body, appropriate muscles have to work for supporting the body structure at the right time, and the muscles that support the body structure have to reeducate the proper function in the right way. This embodiment can be provided the correction of the body by enhancing the function that the muscles originally have, and by improving balance in between the muscles without adding excessive force such as compression or traction.

The protection member 100 can be provided effective support for the muscles by the structure having taping mechanism, and can be provided to reeducate the muscles that have not been effectively used. In addition, the protection member 100 can be provided the proper body management by heightening the correlation in between each muscle. Therefore, the corrected body shape is maintained even after the detachment by activating the muscles in the right place.

### (d) Artificial Hand and Artificial Leg

The protection member 100 may be applied to an artificial hand and an artificial leg. By producing the protection member 100 to have a type directly attached to the skin or a type that uses a material having a high coefficient of friction with the skin, a shift caused by movement is less likely to occur, and friction that may occur at this time can be minimized.

The artificial arm and leg are mounted to compensate for the body parts that have lost, and protection of the attachment parts is the minimum requirement. Bandage or cushioning material may be used to protect a body site at the time of wearing, but the rubbing caused by friction and displacement due to the movement may be produced.

The protection member 100 according to this embodiment can be mounted to the body directly, and may be covered with the layer of elastic material. Therefore, the artificial limb does not need to put extra shock absorber, and the close contact surface 30 can be minimized the displacement due to the movement. The shape defining portion 24 can be modified to fit the body shape, and elastic layer that covers on the shape defining portion 24 can be alleviated the discomfort of wearing.

In addition, this embodiment can enhance the function of the existing part of the body by adding taping structure and ramification structure, and smoothen the functional correlation between the artificial arm (foot) and the existing part of the body. By adding the layer including the cooling material or a heat retention material to the protection member 100, cold or heat problems at the distal end of the body such as poor circulation or stuffiness can be resolved.

### (e) Shoes

The protection member 100 may be configured to be integrated into shoes. Shoes themselves may be configured by the protection member 100, or the protection member 100 may be mounted to shoes to be integrated into the shoes.

The conventional products need to have an extra space in the shoes for moving freely without tightening a foot too much. However, this space produces friction between foot and shoe, caused the problem such as blister and callus formation.

By providing the close contact surface 30 inside of the shoe, and filling the space with elastic material, the protection member 100 covered with elastic layer can be produced a margin that can move freely and can reduce stress due to friction simultaneously. The shape defining portion 24 can be supported the structure of the foot, and can also be functioned for a shock absorption. By covering the protection member 100 with elastic material, a sense of discomfort at the time of wearing is reduced. The protection member 100 with taping structure can be enhanced the functional support, and can also be provided the support in a wider range in the shoes with a wider ground contact surface to the skin such as boots and high cut shoes. By the existence of the close contact surface 30, the foreign substances such as pebbles can also be prevented from entering the shoes. In addition, this embodiment can be provided less friction in the shoes with high contact area of the foot such as five-fingered shoes, and stability to the shoes with small contact area of the foot such as sandals. The shoes having only sole can be produced. By adding the ramification structure, this embodiment can be provided for further support.

By adding the thermal retention material, this embodiment can be warned the body parts effectively in a cold season, and can be prevented functional impairment due to cold by using for the ski boots or trekking shoes.

For the shoes having unstable structure including high-heeled shoes, this embodiment can be provided stability by adding ramification structure or strap structure to the product. By adopting the protection member 100 to shoes for ballet or dance, this embodiment can be reduced the burden to the foot, and can be prevented the possible injury.

### (f) Insoles of Shoes

The protection member 100 may be applied to the insoles of shoes. Accordingly, gaps between the foot and the shoes are filled with the impact relaxing portion 10 of the protection member 100 and thus a sense of unity like a part of the body is generated. In addition, by the taping function of the protection member 100, linkage between muscles is increased, and stability can be increased at the same time.

Typically, when fitting in shoes is performed, a margin of about 1 cm is provided in the toe part so as not to impede the movement in the shoes. On the other hand, the space causes an unnecessary movement and produces the cause of friction. By filling the space of the toe with the protection member 100, friction or impacts are relaxed without impeding the movement in the shoes.

When this function is employed by high-cut shoes, boots or the like, a wider range of supporting is possible.

The conventional products are used by only putting or sticking in the shoes, and may not be considered about influence on the body in friction and displacement that occurs in walking or running. To slip off from a target part can be ineffective to the support area, but also can become a negative effect on unnecessary parts.

However, by attached the protection member 100 directly to the body, this embodiment can be prevented the friction and displacement due to body movement, and can support the target area continuously. In addition, this embodiment can be provided continuous support to the body, since the mounted state will be continued even after taking off the shoes.

This embodiment may have the wide usable range from the product covering up the whole sole to the product protecting only the limited part such as blister or callus. By adopting the soft material to the portion in contact with the affected area, the loads to the affected area and the discomfort when mounting can be reduced. For example, this structure can be adopted to a doughnut pad for the protection of blister.

In addition to a shock absorption function and prevention of friction or displacement, this embodiment may be provided continuous change in the height in the appearance after taking off the shoes by mounting the secret insole (insole for changing the height by lifting up the heel) as a secondary effect.

### (g) Various Protectors

The conventional products protect a body from an external impact by attaching to each target area. The size and thickness of the protector has to become bigger to make the support strong. Furthermore, in order to fit to the shape of the body, the conventional products is used the equipment such as straps for the purpose of prevention of displacement.

For increasing the ability of support, this embodiment can be reduced the burden to the body by stacking a shock absorber and cushioning material, and can be covered the support area accurately by close contact to the body. By directly attaching to the body, this embodiment can be prevented from displacement and incomplete mounting due to a difference in individual body frame. In addition, metal fittings and straps may be removed from the products. By the existence of the close contact surface 30, this embodiment can be mounted any part of the body without any limitation, and can be used for the body part having wide range of motion and uneven surface of the body.

By directly attaching the protection member 100 to the body, a shift is prevented, and incomplete mounting due to a difference in individual body frame or the like can be avoided. Further, unnecessary components such as metal tools and straps can be omitted. A limitation during movement is minimized by separated wearing and thus a degree of freedom can be enhanced. The protection member 100 may be applied to not only innerwear but also outerwear.

In contact sports, protection can further be strengthened by increasing the number of layers of elements of the protection member 100, adding an air layer, and the like. The protection member 100 may also be employed by shoulder pads, hip pads, rib pads, coccygeal pads, neck pads, elbow pads, knee pads, foul cups, earmuffs, large pads, shin guards, helmets, and the like. In addition, the development of a new protector can be accelerated. By using a material having sufficient flexibility, the protection member 100 can be used for a body site with wide range of motion. Since the protection member 100 is provided with the close contact surface 30, the taping function is provided, and thus both effects of protection and function improvement can be expected.

For the body part that has a wide range of motion and for the object that is likely to shift from the target area by external impact, this embodiment can be provided further support by adding the ramification structure.

As a protection for the existing injury such as contusion, by adopting the soft material to the portion in contact with the affected area, the burden caused by contacting the protector to the affected area, and the external impact to the affected area can be reduced. For example, as illustrated in FIG. 1A, by mounting the doughnut pad that the center part is filled with a soft material so as to cover the affected area, the prevention of deterioration of the injury and reducing the discomfort when mounting can be accomplished. Furthermore, as illustrated in 1B, the impact can be dispersed by the bridge part of the deformation suppressing member 20 while ensuring the impact absorbing function of the impact relaxing portion 10, and thus the impact is less likely to reach the part to be protected.

### (h) Supporter with Cooling and Heat Retention Functions

The protection member 100 can be provided with an accommodation portion capable of accommodating a cooling material or a heat retention material, and the protection member 100 may be used as a supporter. Even in a site having irregular unevenness such as the ankle or a site having a wide movable range such as the shoulder, by using the protection member 100 formed along the shape thereof and accommodating the cooling material or a heat retention material therein, cryotherapy and thermotherapy are efficiently performed.

In addition, by attaching the protection member 100 to a site (affected area) to be protected, a shift or a drop due to movement does not occur, and a bandage used to fix the protection member 100 to the affected area is unnecessary. By providing the cooling material or the heat retention member inside the protection member 100, the cooling material or the heat retention member does not directly come into contact with the skin, and thus a risk such as frostbite or low temperature burns can be avoided. As the material of the cooling member or the heat retention member, in addition to a material that can be heated by a microwave or the like, a material that emits far-infrared rays or a material having a heat accumulating action may also be employed. By providing the cooling material or the heat retention material to be replaceable, a plurality of applications can be achieved by the single protection member 100. In addition, by employing a separable structure, without cooling or warming the entirety of the protection member 100, only the cooling material or the heat retention material may be taken out to be warmed or cooled.

This embodiment can be used for one of the treatment tools by using a cold material and a heat retention material. By the existence of the close contact surface 30, this embodiment can be utilized while moving or during other activities without fixing ice packs to the treatment area by bandages and the like. By adopting the separable structure to core layer, cold material and heat retention material can be taken out for being warmed or cooled to the purpose temperature. By replacing the core layer to bag like structure that can be accommodated ice or hot water, the procedure for being warmed or cooled can be omitted, and the use in the whereabouts becomes easy.

### (i) Ankle Weight and the Like

The protection member 100 may be applied to an ankle weight or a wrist weight. By implementing the ankle weight or the wrist weight using the protection member 100, the protection member 100 does not need to be mounted by allowing a joint to go around, and a load is applied without the limitation to the movable range of the joint. Therefore, applying a load to only a limited range can be avoided. Since the weight itself can be attached to the affected body parts, the weight can be mounted on not only the distal parts such as the wrist or the ankle but also parts close to the center of the body such as the arm or the trunk. By the protection member 100 having the taping structure, a load is applied along the structure of muscles and thus biased load can be avoided.

Ankle weight and wrist weight are mounted by allowing a distal part of the body such as wrist and ankle to go around. These products are fixed with the straps with metal fittings or the hook-and-loop fastener, but the conventional products may not have sufficient ability to hold at the attachment site. In addition, these products may be produced friction and a sense of discomfort at the time of the wearing because of partial tightening. To attach a heavy object to the distal part of the body is not be effective for the body mechanics.

By adopting the protection member 100 to ankle weight and wrist weight, this embodiment can be used for trunk and/or shoulder, other than ankle or wrist, since the protection member 100 is attached directly to the body. The shape defining portion 24 can be changed to fit the body shape, and can be prevented from fiction at the time of wearing. By covering the protection member 100 with elastic material, a sense of discomfort at the time of wearing is minimized. Since the protection member 100 does not need to be mounted by allowing a joint to go around, a load is applied without the limitation to the movable range of the joint. By molding the substance with weight along the taping structure, the protection member 100 can be provided the support for the movement of the body, while the protection member 100 can be provided the load to the muscles that work for the movement of the body.

### (j) Seat of Vehicle

The protection member 100 may be applied to the seat of a vehicle. According to the seat of the vehicle, the seat is curved to come into close contact with the body and stabilize the body, and can reduce an unnecessary load on the body. By using the protection member 100 to be used the taping like structure between supported parts from the neck, the lower back to the hips, positional stabilization of the supported parts is accelerated and thus the body structure can be efficiently supported. By adding the ramification structure, this embodiment can be provided for further support.

### (k) Bags

The bags with the protection member 100 can be minimized the motion of the bag when walking. The shoulder strap with layered structure can be decentralized the pressure of the weight of the bag at the specific point by fitting the uneven surface of the body. The part of the backpack corresponding to the back with the close contact surface 30 can be provided the further stability. The elastic material on the surface of this embodiment can be minimized the unnecessary motion of the bag when walking by changing to fit the body shape.

### (l) Grips

Basically, the conventional grips may have the hard surface. The conventional grips with uneven shape are not produced for fitting the shape of the individual hand and fingers. For the long-time use, this product can be lead to tiredness of hand, shoulder and neck. (the grip with the hard surface can be caused the straining of the distal parts).

By adopting the grips with the protection member 100, the shape defining portion 24 can be improved the stability by fitting to the shape of the individual hand without extra effort to gripping, and the stability can be reduced the burden to the hand and the other body parts. Since the existence of the protection member 100 is required less labor in the action of gripping, straining and fatigue are reduced.

### (m) Gloves

As for the purpose of use of the conventional gloves, the prevention of slippage when gripping object, the protection from friction, the protection of first at the time of punching, and/or the protection from external impact can be considered. In ease of adopting the protection member 100 to the gloves, the arrangement and thickness of the protection member 100 can be different by sports or works to be used, and the reinforcement of the protective layer, the addition of the cushioning material, and the removal of an unnecessary parts by the existence of the close contact surface 30 may be considered, if necessary.

### (n) The Bedding

In case of adopting the protection member 100 according to this embodiment, the shape defining portion 24 can be transformed into desirable shape by the load that adds to the shape defining portion 24 (can be changed the shape by the body positioning such as side lying position and supine position due to the load applied at the time). Since the protection member 100 is reduced the load applied to one specific part, the prevention of bedsore and reducing the burden to lower back can be accomplished. The close contact surface 30 with elastic material can be provided the subduction moderately, leading to further stability. By adding the layer of the cold material or heat retention material, seasonal temperature regulation may be accomplished. This embodiment can be used for various products such as pillow, the mattress, and the like.

### (o) Floors

By adopting this embodiment to the floor of bathroom or bathtub, the protection member 100 can be prevented from fall by the close contact surface 30 made of a material having a high coefficient of friction. In addition, this embodiment can be provided the subduction moderately by weight, and the stability at the time of walking. This embodiment may also be used for the place such as stairs that may be tripped over. Rather than wearing the products that are superior in shock absorption, by adding the shock absorption capacity to the ground itself, this embodiment may be reduced the burden to the body at all times.

### (p) Swimsuit, Wet Suit

By adopting the protection member 100 to swimsuit or wetsuit, this embodiment can be provided the compression moderately for high-performance swimsuits, and the improvement of body function by adding taping structure. The conventional wetsuits may have difficulty of putting on and taking off. By adding the close contact surface 30 or the function of separated wearing, the products may become easy to put on and take off. The existence of the close contact surface 30 can be prevented from the ingress of water to inside of the wetsuit without fastened tightly (to fasten tightly is the one element for making it difficult to wear). Furthermore, the protection member 100 can be provided to maintain body temperature effectively in the cold season by adding heat retention material.

### (q) Glasses, Goggles

The protection member 100 can be adopted for the parts of nose pads, the parts of ear handle of glasses, and the like. This embodiment may be omitted the part of glasses depending on an overall shape. By adopting the protection member 100 to the parts that are contacted with face, this embodiment can be reduced the possibility of injury caused by glasses or goggles when a fall or hitting by foreign object.

### (r) Training Equipment

By covering a dumbbell with the protection member 100, this embodiment can lower the possibility of the injury caused by the fall of the dumbbell. The grip with protection member 100 can be reduced the burden to the hand. This embodiment can be provided the stability for heightening the training efficiency by adopting to the seat, the back, the pad and the like. Besides, this embodiment can be adopted to a balance ball, a stretch pole, yoga mat, a medicine ball, a tube and the like.

### (s) Barrier-Free Related Products

By adopting the protection member 100 to handrail, the floor in the bathroom or stairs with the possibility of slippage, this embodiment can be avoided the danger from the fall. The protection member 100 can be provided the continuous stability of the body by transforming into the shape to grasp firmly with a weak muscular strength. By adopting to the manual operation part of the wheelchair, the handle of the cane and the part of the cane contact to the ground, the part under the armpit of the crutches and the like, this embodiment is increased the stability with minimum effort, and is reduced fatigue. By fitting the shape of the hand to compensate for a weak muscular strength, the protection member 100 can be provided the firm gripping when open and close the lid of the bottle without borrowing somebody's hand.

### (t) Triangle Bandage, Piggyback String

By adopting the protection member 100 to triangle bandage or piggyback string, this embodiment can be reduced the burden to the user by having the close contact to the body. The triangle bandage can not only hang the arm, but also can be reduced vibration and a pain with the movement of the body by adopting the close contact surface 30. The piggyback string can be reduced vibration with the movement of the body and the burden on user by fixed firmly to the body when holding the baby in front or on the back. In addition, the center of gravity is shifted to near the body by the close contact to the body and is reduced the burden to the lower back of the users. By adding the ramification structure, this embodiment can be provided easy adjustment for the stability.

### (u) Mouthpiece

The conventional mouthpiece is molded by their own and may be inferior to the made-to-order products in function and accuracy. The made-to-order products may take time to mold, and may increase the costs.

By adopting the protection member 100 to mouthpiece, the shape defining portion 24 can be deformed the shape according to the individual, and can be dispersed the pressure. This embodiment can be accommodated the individual difference without molding by the made-to-order. By the existence of the close contact surface 30, this embodiment can be mounted to the target site accurately, and can be provided the continuous protection against the sudden impact without departing. Furthermore, this embodiment can be prevented from the shift in a mouth, and can be provided an easy breathing by reducing the sensation of foreign object in the mouth.

The conventional mouthpiece is provided the protection of upper and lower teeth by one mouthpiece. By the existence of the close contact surface 24, the separation type of mouthpiece for upper teeth or for lower teeth can be considered. Accordingly, the opening and closing of the mouth can be accomplished easily.

### (v) Earphone, Earplugs

The conventional earphone and earplugs are adopted the shape recovery material for fitting the shape of the individual earhole, but are not sufficient to fill the gap completely. In addition, since the material with a little friction with the skin is used, the products can be easy to come off, and can be strongly felt the sensation of foreign object in the ear. The hard material can be used for the earphone without consideration of the sensation to the ear.

However, by adopting the protection member 100 to earphone and earplugs, the layers such as the shape defining portion 24 or the base body 22 can be provided the stability to the structure, and the elastic material (material that has near flexibility of the skin) can be increased the degree of the close contact. By the shape of the shape defining portion 24 or the base body 22, and by the existence of the close contact surface 30, this embodiment can be provided the minimum shift from the mounted site. By the existence of the close contact surface 30, this embodiment can be adopted the earphone of the bone conduction type that can be stuck to the skin. This embodiment can be reduced the parts to be used for the earphone of the ear hanging type. The conventional products may be covered with the protection member 100.

### (w) Massage Chair, Massage Device

The conventional massage chair and massage device have the functions such as compression, kneading, and vibration. By adopting the protection member 100, this embodiment can be added the function for stretching by increasing the degree of the close contact (massage balls of massage chairs fix two points and move to separate the two points. Accordingly, the muscle between two points is stretched out).

By adopting the protection member 100 to the massage balls as well as the bearing surface of the massage chair, this embodiment can be provided softer stimulation to the body, and subtle intensity adjustment (the stimulation to the body by the hard surface of the massage ball can be stronger despite a slight difference of pressure). By adopting the protection member 100 to the surface of other massage devices, this embodiment can be provided better sensation to the body.

### (x) Bulletproof Vest, Stab Vest

By adopting the protection member 100 to bulletproof vest and stab vest, this embodiment can be provided to not only protect the body from a bullet and a knife, but also to alleviate the impact at the time of attacking. This embodiment can be prevented from the penetration of the bullet, and can be dispersed the impact by overlapping of plurality of layers (layer of shock absorber and the cushioning material), and thus the impact is less likely to reach the body. By adopting the refractory fibers or the like, this embodiment can also be utilized for the fireproof suit or explosion proof suit.

### (y) Artificial Joint

Since an artificial joint is to be installed inside of the human body, the materials that have a long service life can be used. The artificial joints to be used currently have the characteristic of a solid, rust durable and smooth movement of the joint, but the function of the shock absorption may not be considered that far.

Rather than the burden is added to the artificial joint itself, the burden is increased in the ground part of the artificial joint and the existing bone. By adopting the protection member 100 to the internal structure of the artificial joint, this embodiment can be reduced the burden to the body. In addition, by adopting the fibrous material with a long service life such as carbon nanotube, this embodiment can be used on the articular surface, and the ground part of the artificial joint and the existing bone. The protection member 100 may be interposed between the bone and the artificial joint.

### (z) Sandbags

By adopting the protection member 100 to sandbags, this embodiment can be provided the protection of a first or leg of the user. By adopting the protection member 100 to the gloves or shin supporter to be wearing, this embodiment can be achieved the bidirectional shock relaxation. By stabilizing the structure, this embodiment can be reduced the impact with the blow and the impact caused by repulsion.

### (α) Belt of the Watch, Heart Rate Monitor

By adopting the protection member 100 to belt of the watch and heart rate monitor, the belt of the watch may not be necessary to go around a body part, and can be mounted selectively such as dorsal surface, palmar surface, or ulnar aspect of the wrist. In the heart rate monitor, this embodiment can be provided the easy mounting, and can be reduced uncomfortable sensation associated with the body movement.

Since this embodiment is attached directly to the body by the close contact surface 30, the development of a new product other than the shape that currently exists can be accelerated.

### (β) Electronic Equipment Such as Cell-Phone, PC, and the Peripheral Device

In the usage of the electronic equipment such as a cell-phone, a PC, and the peripheral device, the burden on the body is increased by performing the same operation repeatedly. In addition, the burden to the joint is increased depending on the posture or the position of the body. By adopting the protection member 100 to the pad that adjusts the wrist angle, this embodiment can be prevented from the tenosynovitis of the wrist.

By adopting the mouse or keyboard, this embodiment can be reduced the burden caused by the repeated use. As a secondary effect, this embodiment can be effective for the shock relaxation from the external impact in the electronic device which is mounted this structure.

### (γ) Pet-Related Supplies

This embodiment can be used for collar, harness for dogs, shoes for dogs (the use of adhesive material for pads), the handle of the lead, wheelchair for pets, and harness for horses. To pull (or to be pulled) the collar or dog harness can become the burden to both pets and owners. By adopting the protection member 100 to pet-related article, this embodiment can be reduced the burden of such. This embodiment can also be reduced the burden at the time of horseback riding by adopting to the harness. Thermal insulation sheets for horses, wet suit for dogs and the like can be considered.

### (δ) Beauty Supplies

By adopting the protection member 100 to face masks, face roller, and the like, this embodiment can be useful to give the stimulation softly without the damage to the skin. As with the other muscles of the body, the muscles of the face can be maintained a toned and lifted condition under the proper use of the muscles, and the face masks that is adopted the taping structure can become effective. This embodiment can be achieved the role such as a facial pack simultaneously by adding the layer including the cosmetic ingredients.

### (ε) Tapes for Taping

By adopting the protection member 100 to the tapes for taping, this embodiment can be provided the support to the muscles and the improvement of the muscle function effectively. The disposable type and the reusable type of tape can be developed depending on the members to be used.

By adopting the structure with the curve shape of the outer edge of the tape, and thinner outer edge compared to the other part of the tape, this embodiment can become hard to come off and can be provided the continuous support of the body effectively.

### (ζ) Medical Supplies

Medical supplies such as bandage, a triangle bandage, a neck color, and a plaster cast are used very often in the onsite of medical setting. By adopting the protection member 100 to medical supplies, this embodiment can be provided the support accurately without shifting from support area, and can also be enhanced the recovery in addition to the pain relief.

For the medical supplies such as plaster cast that have a difficulty of taking off after mounting, this embodiment can be provided easy adjustment for fixation, protection, and putting on and taking off at any time, and can be eliminated the effort to reconstruct the plaster cast for the adjustment of error that occurs through the recovery process.

### (η) Seat Belt

By adopting the protection member 100 to the seat belt, this embodiment can be reduced discomfort at the time of mounting, and can be provided the protection of the body from the impact at the time of the accident. By wearing the seat belt, this embodiment can be avoided the life threatening situation, and can also be reduced the damage to the body caused by the seat belt itself. By adding the ramification structure, this embodiment can be provided for further support.

### (θ) The Concepts of the Other Applied Examples

(i) By adopting the protection member 100 to the object to add the impact to the body, or the object that may have possibility to add the impact to the body, this embodiment can be protected the human body from the impact to be added. In addition, by changing the structure of the object itself, this embodiment can be reduced the impact from the object to add the impact or the objects that may have possibility to add the impact to the body. By adopting the protection member 100 to the ball or the body of the car, this embodiment can be reduced the impact when collided by any chance.

(ii) By stabilizing the structure of the object itself, this embodiment can be reduced the burden on the user. The protection member 100 can be used for grips, sand bags, and the like. Due to stabilize the structure of the object to be used, this embodiment can be reduced the load on the body since the user may not be necessary to put extra effort when using. As for the products such as sand bags that aim to provide the impact to the object by user, this embodiment can be alleviated both the impact to the body caused by the blow and the repulsive force that is rebounded to the body by contact with the object.

(iii) By adopting the protection member 100 to the products that may have possibility of collision in the sports setting, this embodiment can be reduced the impact to the body. The protection member 100 can be used for goalpost or hurdles for track and field.

(i v) For safety issues, the protection member 100 can be adopted for the object that may have the function of shock absorption, and the ability of change and restoration of the shape. This embodiment can be prevented from injury by changing the shape of the object such as toys that the child uses. Since the deformation suppressing member 20 can be restored to its original shape by adopting the shape memory material, this embodiment can be minimized the possibility of damage to the product. In case of being accidentally hit the body such as a face, this embodiment can be dispersed the impact by changing the core shape. By adopting the soft material to the surface layer, this embodiment can be reduced the impact at the time of hitting. As a specific example for adopting the protection member 100, the objects such as a dodge ball, imitation sword, and toys like figures that are generally used by child can be included. In addition, by adopting the protection member 100 to the part of the object such as a corner of a desk that is likely to hit by a person, this embodiment can be reduced the risk of sudden accidents. As a secondary effect, this embodiment can be strengthened the moving parts of the object, and can be increased the durability by covering the object such as figure that consists of multiple parts with the protection member 100.

### (ι) Belt, Straps of Harness

The belt or the straps of the harness can be used for increasing the stability by tightening the part of the body, and the tightness caused by the muscles contraction associated with the movement is provided further compression to the mounted part. In addition, the products with serious consideration to safety such as harness may be required stronger tightening to the mounted part. For reducing the discomfort caused by tightening, and for ensuring the safety when mounting, the member to be used may be necessary to be stretched to some extent in accordance with the muscle contraction without disturbing the muscle action. The deformation suppressing member 20 can be maintained the shape of this embodiment, and the impact relaxing portion 10 covering the deformation suppressing member 20 can be reduced the discomfort when mounting, and can be stretched with the body movement. In addition, by the existence of the close contact surface 30 for the support of the mounting area accurately, this embodiment can be reduced the friction and the shift with the body movement. The addition of the ramification structure can be provided for further support.

### (κ) Accessories to the Conventional Products (Ready-Made Products)

By adopting the protection member 100 as accessories to the conventional products, this embodiment can be enhanced the support easily, and can be reduced the discomfort at the time of usage. The advantage of applying this embodiment later can be desirable for the products that may be required cost and labor for replacing all. This embodiment can be adopted for the part, or the whole surface of the conventional products. This embodiment can be added to variety of products such as the strap structure in high-heeled shoes, the shoulder strap of the bag, the bearing surface and backrest of the chair, handrails, crutches and the like.

This embodiment can be modified in various forms within a range of the gist of the invention. In addition, the protection member 100 described in this embodiment can complete the role as a protection tool by the protection member itself. In addition, the protection member 100 can also be combined with other protection tools. The protection member 100 can be incorporated electromagnetic devices and a substance capable of generating the magnetic field.

### INDUSTRIAL APPLICABILITY

The invention can be applied as, for example, a support member of the body of a person or an animal.

### DESCRIPTION OF SYMBOLS

10 Impact relaxing portion
20 Deformation suppressing member
20a Projecting portion
22 Base body
24 Shape defining portion
26 Groove
28 Element member of shape defining portion
30 Close contact surface
100 Protection member
200 Contact tool

What is claimed is:

1. A protection member for protecting at least a portion of the body of a person or an animal, comprising:
an impact relaxing portion which is changed in shape according to a shape of a part of the person or the animal to be mounted, and absorbs an impact;
a deformation suppressing member which suppresses deformation of a shape of the protection member and disperses the impact absorbed by the impact relaxing portion; and
a close contact surface which is provided in at least a portion of a mounting surface of the protection member that is mounted on the person or the animal,
wherein the deformation suppressing member includes a base body made of a material harder than the impact relaxing portion and a shape defining portion made of a material harder than the base body.

2. The protection member according to claim 1,
wherein a plurality of base bodies are provided,
the plurality of base bodies are provided to be lined up along a planar direction of the protection member, and
the shape defining portion is provided in at least a portion between the base bodies.

3. The protection member according to claim 1,
wherein the plurality of shape defining portions are provided, and
the shape defining portions and the base bodies are alternately arranged.

4. The protection member according to claim 1,
wherein a cross-sectional shape of the deformation suppressing member is a cantilever shape or a bridge shape.

5. The protection member according to claim 1,
wherein the shape defining portions are configured of element members of the plurality of shape defining portions, and
the element members of the plurality of shape defining portions are arranged in a row.

6. The protection member according to claim 5,
wherein an extension direction of the element members of the shape defining portions is an oblique direction with respect to a thickness direction of the protection member.

7. The protection member according to claim 1,
wherein a groove is provided on a surface of the shape defining portion.

8. The protection member according to claim 7,
wherein an extension direction of the groove is an oblique direction with respect to a thickness direction of the protection member.

9. The protection member according to claim 1,
wherein the deformation suppressing member is made of a material having a heat retention or a cooling function.

10. The protection member according to claim 1, further comprising:
an accommodation portion which accommodates a cooling material or a heat retention material.

11. The protection member according to claim 1,
wherein a projecting portion is provided to protrude outward from the impact relaxing portion.

12. A contact tool provided with the protection member according to claim 1 on a surface.
